(19) United States
(12) Patent Application Publication
(10) Pub. No.: US 2018/0325574 A1
(43) Pub. Date: Nov. 15, 2018

(54) SWIVEL DRIVE SYSTEM FOR SCREW INSERTION

(71) Applicant: Spineology Inc., St. Paul, MN (US)

(72) Inventors: Todd BJORK, Hudson, WI (US); Lauren Berger VANBEEK, St. Paul, MN (US); Adam SHINBROT, WOODBURY, MN (US); Steve JACOBSON, Lake Elmo, MN (US)

(21) Appl. No.: 15/942,405

(22) Filed: Mar. 30, 2018

Related U.S. Application Data
(60) Provisional application No. 62/480,156, filed on Mar. 31, 2017.

(51) Int. Cl. A61B 17/88

(52) U.S. Cl. CPC A61B 17/8875; A61B 2017/00477

(57) ABSTRACT

A swivel screw driver including an articulating ball joint mechanism. The ball joint mechanism may allow for up to the range of 40° of angulation. The ball joint mechanism provides for 360° of rotation while driving a distal tip. The driver can include a drive member, a ball disposed at least partially within the drive member, the ball including a drive pocket defined therein, a pin extending through the drive pocket and restrained on opposing longitudinal ends thereof to the drive member such that the pin can only rotate about its longitudinal axis, and a guide body connected to the ball.

### PRIORITY

This application claims the benefit of U.S. Provisional Application Ser. No. 62/480,156, filed on Mar. 31, 2017, which is hereby incorporated herein by reference in its entirety.

### FIELD

The present invention generally relates to a swivel drive for screw insertion. More particularly, the present invention relates to a screw driver having a swivel drive with angle maintaining tension that may be used for placing surgical implants.

### BACKGROUND

It is desirable to provide a screw driver capable of angulation to insert screws into a patient at various surgical trajectories. Angled screw insertion allows the surgeon to minimize the surgical access opening and reduce disruption to the patient's anatomy. Presently available devices, such as spring drive joints and socket swivel joints present exposed mechanisms which may entrap patient's tissue when in use. Also, joint mechanisms exist that provide for rotation and angulation under tension, but none that also allow driving. Therefore, improved screw drive mechanisms are needed.

### SUMMARY

The disclosure includes the a dive joint including an articulating ball joint mechanism. In an example embodiment, a ball joint mechanism may allow for up to the range of 40° of angulation. According to a particular aspect, a ball joint mechanism provides for 360° of rotation while driving a distal tip.

The articulating ball joint mechanism may be spring-loaded, causing tension to control the position of the distal tip. In another embodiment, tension may be achieved by any tensioning mechanism, including but not limited to: springs, plastic bushings and/or rubber. In an embodiment of the present invention, the tension delivered by the spring or other tensioning mechanism, may allow a driver tip to remain in any position along and within its 40° of angulation and 360° of rotation during insertion and removal, while still allowing for full rotation and driving of the joint. Although the driver tip stays in place, it is able to be moved and driven easily without a locking mechanism.

In an embodiment, the driver tip may be a screw driver tip. In another embodiment the driver tip may be a drill tip. Any driver tip may be used with the swivel driver of the present invention.

According to one aspect, a spring-loaded drive joint allows a drive tip to be positioned at any angle up to 40° and stay in place during insertion and removal. Although the tension of the spring maintains the tip's position, the mechanism can be driven easily without any additional steps (e.g. no locking mechanism).

The drive joint device can be sized much smaller that currently available devices, providing better visibility past the joint.

The disclosure includes a spring tension mechanism which allows a drive tip to remain in place upon insertion into a drill guide, thus preventing premature dropping of the drive tip which could potentially cause bodily harm.

The drive joint that may be completely enclosed, thus protecting the patient's tissue from being caught in the drive joint mechanism.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a cross-sectional view of an embodiment of the drive joint.

FIG. 2 is a perspective view of an embodiment of the drive joint.

FIG. 3 is a perspective view of an embodiment of the drive joint.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims. For illustrative purposes, cross-hatching, dashing or shading in the figures is provided to demonstrate sealed portions and/or integrated regions or devices for the package.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

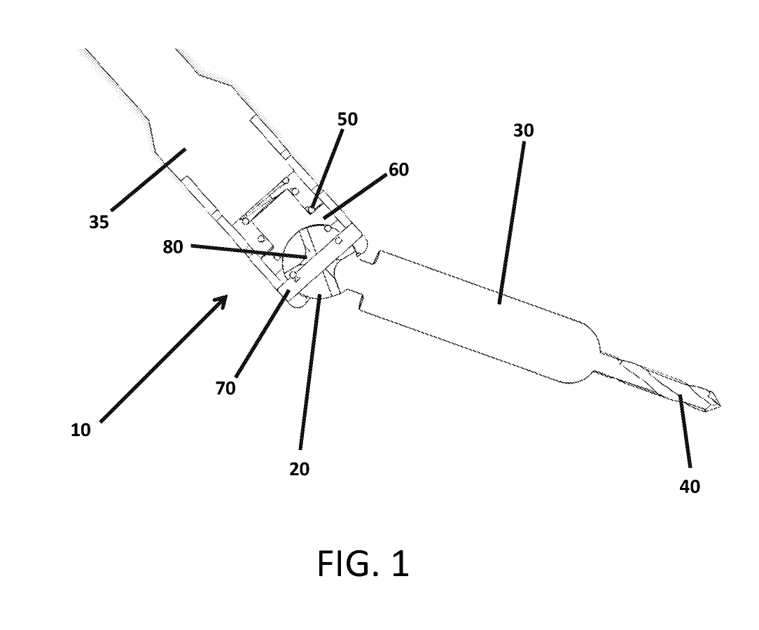

Referring to FIG. 1, swivel driver 10 is depicted. Driver 10 includes a ball 20 that articulates through 40 degrees of angulation and 360 degrees of rotation. Guide body 30 connects ball 20 to tip 40. The ball is partially disposed within drive member 35.

Drive pin 70 moves within a drive pocket 80 defined into the ball 20. The pin 70 is disposed within the drive member and is constrained to only axial rotation.

The drive pocket 80 allows ball 20 to move with respect to the pin 70 through at least 40 degrees of angulation and 360 degrees of rotation. Of course, a different maximum angulation figure can be defined. The drive pocket 80 is sized and shaped to allow the pin 70 to move sufficiently so that the intended maximum angulation can be achieved.

Tension mechanism 50 applies tension to retention mechanism 60, which rotatably retains ball 20 in place. The tension mechanism can be any suitable biasing component, such as a compression spring (as shown), bevel washers, bushings, and compressible materials that will return to their original state when a compression force is removed (e.g. most rubber materials).

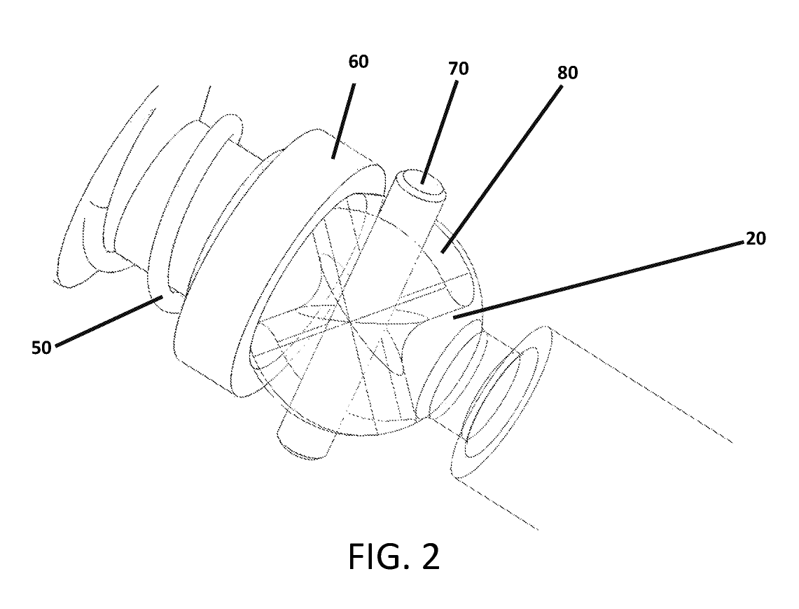

As can be seen in FIG. 2, the drive pocket 80 may be a cutout or slot defined in ball 20 to provide a path for drive pin 70 to drive ball 20 through 40 degrees of angulation and any angulation between 0 and 40 degrees.

In the embodiment shown in FIG. 2, tension mechanism 50 is depicted as a spring and retention mechanism 60 is depicted as a spherical plate. According to this embodiment, a spring loaded spherical plate allows for spring tension to be applied to ball 20 holding tip 40 in position without causing drag during rotation.

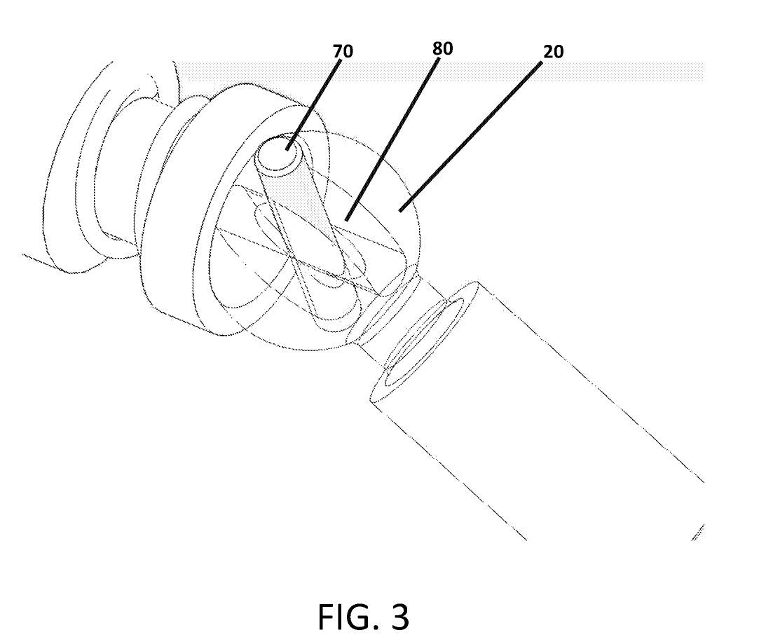

FIG. 3 depicts ball 20 rotated along a different angle than shown in FIG. 2. Drive pin 70 and drive pocket 80 have been rotated to show movement from that shown in FIG. 2.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A swivel driver that articulates through at least 40 degrees of angulation and 360 degrees of rotation, comprising:
a drive member;
a ball disposed at least partially within the drive member, the ball including a drive pocket defined therein;
a pin extending through the drive pocket and restrained on opposing longitudinal ends thereof to the drive member such that the pin can only rotate about its longitudinal axis; and
a guide body connected to the ball,
wherein the drive pocket is sized and shaped to permit the guide body to articulate through at least 40 degrees of angulation and 360 degrees of rotation.

2. The swivel driver of claim 1, wherein the drive pocket is a slot defined through the ball.

3. The swivel driver of claim 1, further comprising a tension retention mechanism to rotatably retain the ball within the drive member.

4. The swivel driver of claim 3, wherein the tension to retention mechanism is a compression spring.

5. The swivel driver of claim 3, wherein the tension to retention mechanism is a spring loaded spherical plate.
